(19) United States
(12) Patent Application Publication
(10) Pub. No.: US 2014/0171415 A1
(43) Pub. Date: Jun. 19, 2014

(54) FUNNY CURRENT (IF) INHIBITORS FOR USE IN A METHOD OF TREATING AND PREVENTING HEART DISEASE IN CANINE

(71) Applicants: Saskia KLEY, Appenheim (DE); Ingo LANG, Ingelheim am Rhein (DE); Joerg Christian MEIL, Weiler (DE); Randolph SEIDLER, Eckenroth (DE); Michael MARKERT, Aulendorf (DE)

(72) Inventors: Saskia KLEY, Appenheim (DE); Ingo LANG, Ingelheim am Rhein (DE); Joerg Christian MEIL, Weiler (DE); Randolph SEIDLER, Eckenroth (DE); Michael MARKERT, Aulendorf (DE)

(73) Assignee: Boehringer Ingelheim Vetmedica GmbH, Ingelheim am Rhein (DE)

(21) Appl. No.: 14/102,622

(22) Filed: Dec. 11, 2013

(30) Foreign Application Priority Data
Dec. 14, 2012 (EP) 12197371.3

(51) Int. Cl. A61K 31/55; A61K 31/136

(52) U.S. Cl. CPC A61K 31/55; A61K 31/136
USPC 514/212.07; 540/523; 514/646; 564/430

(57) ABSTRACT

The present invention relates to an I_{f} blocker or a pharmaceutically acceptable salt thereof for the treatment and/or prevention of a canine patient suffering from heart diseases, preferably heart diseases such as dilated cardiomyopathy (DCM), mitral valve insufficiency (MI), arrthymias, preferably tachyarrthymias, preferably arterial arrhythmias, atrioventricular nodal arrhythmias and/or tachycardia. Each of these diseases may or may not result in heart failure (HF) in canine patients. The invention also relates to improving the quality of life, improving the general health condition as well as a prolonging the life expectancy in canine patients suffering from heart diseases and/or heart failure due to one or more of the following etiologies dilated cardiomyopathy (DCM), mitral valve insufficiency (MI), arrthymias, preferably tachyarrthymias, preferably arterial arrhythmias, atrioventricular nodal arrhythmias and/or tachycardia.

### FIELD OF THE INVENTION

The invention relates to the field of medicine, in particular to the field of veterinary medicine. The invention relates to the effect of funny current (I_{f}) inhibitors or a pharmaceutically acceptable salt thereof for use in a method of treating and/or preventing heart disease, preferably heart diseases such as dilated cardiomyopathy (DCM), mitral valve insufficiency (MI), arrthymias, preferably tachyarrthymias, preferably arterial arrhythmias, atrioventricular nodal arrhythmias and/or tachycardia, each of these diseases may or may not result in heart failure (HF) and/or arrhythmias in canine patients. It further relates to improving the quality of life and the general health condition as well as prolonging the life expectancy in canine patients suffering from heart diseases and/or heart diseases due to one or more of the following etiologies DCM, MI and/or tachyarrhythmia's.

### BACKGROUND OF THE INVENTION

There are many different heart diseases in canine, which can have many different and accumulative etiologies. Canine myxomatous mitral valve disease (MMVD) is the most common cardiac disease in dogs accounting for more than 70% of all canine heart disease (Detweiler D K, 1965; Haggstrom J et al., 2009). Approximately 30% of all dogs that develop MMVD develop mitral regurgitation (MR) and eventually congestive heart failure (CHF). Myxomatous mitral valves are characterized by a disorganization of the structural components of the leaflets and a weakening of the chordae tendineae (CT), which causes the valve to lose its mechanical ability (Grande-Allen et al., 2003). MR ensues, the valve continues to deteriorate, the MR increases, and eventually the left atrium and left ventricle undergo eccentric hypertrophy (dilation). The pressure in the left atrium continues to rise, eventually exceeding the pulmonary venous pressure leading to active CHF (pulmonary oedema).

Canine idiopathic dilated cardiomyopathy (DCM) is in general followed by progressive CHF or sudden death (Calvert 1986; Calvert 1982). Histopathological characteristics of DCM are divided into two forms: the most common attenuated wavy fiber type (Harpster, 1993). The myocardial lesions associated with the attenuated wavy fiber type consist of thinner than normal myocytes, wavy appearance, separated by a clear space (indicating edematous fluid) with diffuse subendocardial fibrosis (Sandusky, 1984; Dukes-McEwan, 2003). The myocardial lesions associated with the fatty infiltration type of DCM include myocytolysis, myofibre degeneration, vacuolization and myocyte atrophy with extensive fibrosis and fatty infiltration (Harpster 1983; Harpster, 1991).

Treatment of heart diseases encompasses many different types of treatment. For example angiotensine-converting-enzyme inhibitors (ACE inhibitors), positive inotropes, anti-thrombotic agents, and bradycardic agents are often administered to dogs suffering from a heart disease.

ACE inhibitors are used to reduce the activity of the renin-angiotensin-aldosterone system. Known ACE inhibitors, which may be used for this purpose are enalapril, ramipril, benzazepril, quinapril, perindopril, lisinopril, imidapril, zofenopril and trandolapril.

Positive inotropes are used if the systolic function of the heart is decreased. Known positive inotropes which may be used for this purpose are pimobendan, dopamine, dobutamine, epinephrine, norepinephrine, isoprenaline, digoxin, digitalis alkaloids, and theophylline.

Anti-thrombotic agents are administered in order to prevent the development of a thrombus or to dissolve it, if it already exists. Known anti-thrombotic agents which may be used for this purpose are antiplatelet drugs such as clopidogrel, aspirin; anticoagulants such as heparin, warfarin, low molecular weight heparin and thrombolytic drugs such as streptokinase, tissue plasminogen activators.

Elevated heart rate, especially in pet animals, may be treated with bradycardic agents such as for example calcium (Ca²⁺) channel blockers, beta-receptor blockers and other antiarrhythmic agents.

Known Ca²⁺ channel blockers, which may be used for this purpose are diltiazem, verapamil, amlodipine and nifedipine.

Other known antiarrhythmic agents, which may be used for this are adenosine, amiodarone, atropine, digoxin, isoproterenol, lidocaine, tocamide, mexiletine, phenyloin, procainamide, propafenone and quinidine, as for example sulfonates gluconates. Calcium channel blockers as well as beta blockers can be used as antiarrhythic agents as well.

Known beta-receptor blockers, also referred to as β-adrenergic blockers, which may be used for this purpose are atenolol, bisoprolol, carvedilol, esmolol, sotalol, metoprolol or propanolol.

β-Adrenergic blocker therapy reduces morbidity and mortality in human patients with CHF (Packer et al., 1996), and is directly related to heart rate reduction (HRR) (Packer et al., 1996; Nagatsu et al., 2000). Dogs with experimentally induced MR, paced at an elevated heart rate, and given a beta blocker do not show improvement in left ventricular (LV) function (Nagatsu et al., 2000). Bradycardia is a major mechanism by which β-blockers are effective for restoration of contractile function in a model of LV dysfunction (Nagatsu et al., 2000). Canine MMVD patients in a late disease state tend to be highly sensitive to beta blockers and most cardiologists avoid the use of beta blockers in dogs at this specific stage of the disease (Atkins et al., 2009). Moreover, the negative inotropic action of drugs such as beta blockers are often associated with adverse effects in patients with DCM (Calvert, 2004). Furthermore, this negative inotropic effect is thought to lead to the deterioration of canine patients with an advanced heart disease (Atkins et al., 2009). A recent veterinary study used a beta blocker in attempts to delay the onset of CHF in dogs at a specific disease state, the patients were started on a beta blocker or placebo and followed until signs of CHF developed. However, the study did not support the clinical efficacy of the beta blocker to delay CHF in dogs with MMVD (Keene et al., 2012).

Treatment of heart diseases may also encompass the use of all types of diuretics such as loop diuretics, thiazides, carbonic anhydrase inhibitors, potassium-sparing diuretics, calcium-sparing diuretics or osmotic diuretics. These can be used alone or in combination with any of the above mentioned therapies. An example of a diuretic is furosemide.

Thus, the use of many drugs remains controversial since a beneficial effect on disease progression or survival has not been demonstrated with any drugs. So far known treatments in dogs result in temporary relief of symptoms mainly with a diuretic drug to prevent further decompensation of heart failure. However, all of these therapeutic regimens are only of supportive character and therefore limited.

This clearly demonstrates that there is a need to develop a treatment and method of preventing heart diseases in canine, in particular dogs, and to improve the state of the disease, morbidity, quality of life and long-term survival time, especially in canine, in particular dogs, suffering from heart diseases of any etiology.

Thus, the problem underlying the present invention is to provide a medication, which allows the treatment of heart diseases with or without heart failure in canine patients, in particular dogs. Further, it was desired to find a way to improve the disease state, quality of life and to reduce the risk of death in canine patients, preferably dogs, in particular in those suffering from heart diseases of any etiology.

### DESCRIPTION OF THE INVENTION

Before the embodiments of the present invention it shall be noted that as used herein and in the appended claims, the singular forms “a”, “an”, and “the” include plural reference unless the context clearly dictates otherwise. Thus, for example, reference to “a preparation” includes a plurality of such preparations. Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was omitted from the description. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

The solution to the above technical problem is achieved by the description and the embodiments characterized in the claims.

The invention relates to a funny current (I_{f}) inhibitor or a pharmaceutically acceptable salt thereof for treating and/or preventing of heart disease(s), preferably related to heart disease(s) in canine patients, preferably dogs. Furthermore, the invention relates to a funny current (I_{f}) inhibitor or a pharmaceutically acceptable salt thereof for improving quality of life and/or improving general health condition and/or prolonging life expectancy in canine patients, in particular dogs, suffering from heart disease(s).

The term “heart disease” or “heart diseases” as used herein relates to a condition in which a problem with the structure or function of the heart impairs its ability to supply sufficient blood flow to meet the body's needs, in particular any contractile disorder or disease of the heart. Heart diseases may be caused by for example one or more conditions, which may be acquired naturally occurring valvular diseases and/or acquired or naturally occurring diseases of the cardiac muscle. Clinical manifestations are as a rule the results of changes to the heart's cellular and molecular components and to mediators that drive homeostatic control. When the heart disease progresses many dogs experience murmur of valvular regurgitation before the clinical onset of heart failure. There are many different causes such as dilated cardiomyopathy (DCM), mitral insufficiency (MI), arrhythmias, and tachycardia can individually or combined result in heart failure. Thus, the funny current (I_{f}) inhibitor or a pharmaceutically acceptable salt thereof is used for treating and/or preventing heart diseases comprising or consisting of dilated cardiomyopathy (DCM), mitral valve insufficiency (MI), arrhythmias, preferably tachyarrthymias, preferably arterial arrhythmias, atrioventricular nodal arrhythmias and/or tachycardia. According to a further aspect the present invention relates to a funny current (I_{f}) inhibitor or a pharmaceutically acceptable salt thereof for treating or preventing heart failure, preferably due to one or more of the following etiologies DCM, MI, arrhythmias, preferably tachyarrthymias, preferably arterial arrhythmias, atrioventricular nodal arrhythmias and/or tachycardia. Tachycardia as used herein also relates to supra and/or ventricular tachycardia. A further aspect of the inventions is the treatment or prevention of arrhythmias including tachyarrthymias, arterial arrhythmias and atrioventricular nodal arrhythmias associated with or leading to underlying myocardial/heart diseases such as DCM and MI.

Thus, the invention also relates a funny current (I_{f}) inhibitor or a pharmaceutically acceptable salt thereof for improving quality of life and/or improving general health condition and/or prolonging life expectancy in canine patients, preferably dogs, suffering from a heart disease as defined above and/or suffering from HF due to one or more of the above defined etiologies and/or arrhythmias as defined above. The invention also relates to the management of heart diseases comprising or consisting of DCM, MI, arrthymias, preferably tachyarrthymias, preferably arterial arrhythmias, atrioventricular nodal arrhythmias and/or tachycardia; or to the management of heart failure in dogs due to DCM, MI, arrthymias, preferably tachyarrthymias, preferably arterial arrhythmias, atrioventricular nodal arrhythmias and/or tachycardia. According to another aspect the invention relates to the management of arrhythmias associated with underlying myocardial diseases such as DCM and MI.

The degree of the heart disease in canine patients, in particular in dogs, is classified by the New York Heart Association (NYHA) and International Small Animal Cardiac Health Council (ISACHC). According to this classification scheme HF can be classified into four stages:
Class I: Heart disease is present, but no clinical signs are evident even with exercise.
Class II: Heart disease is causing clinical signs only with strenuous exercise.
Class III: Heart disease is causing clinical signs with routine daily activities or with mild exercise.
Class IV: Heart disease is causing severe clinical signs even at rest.

More recently, and as described in the 2009 ACVIM consensus statement (Atkins C, Bonagura J, Ettinger S, Fox P, Gordon S, Haggstrom J, Hamlin R, Keene B, Luis-Fuentes V, Stepien R., Guidelines for the diagnosis and treatment of canine chronic valvular heart disease. J Vet Intern Med 2009; 23:1142-1150) a second type of assessment/classification has been developed to stage HF, which emphasizes more the progressive nature of most of the diseases that cause HF:
Stage A: Includes patients that are at high risk of developing a heart disease and are asymptomatic.
Stage B: The Patients have structural heart disease such as murmur of mitral valve regurgitation or show beginning of a left-sided enlargement, but have never developed clinical signs of heart failure. Due to the non-clinical signs at this stage the following sub-division is made:
Sub-stage B 1: Asymptomatic patients with no evidence of cardiac remodeling during echocardiography or radiographic examinations.

Sub-stage B2: Asymptomatic patients with haemodynamically significant valve regurgitation as seen by echocardiography or radiographic examinations (left-sided enlargement is visible)
Stage C: Patients with past or current clinical signs of heart failure associated with structural heart disease
Stage D: Patients have end-stage disease with clinical signs of HF.

Thus, as an example, a dog that for example belongs to class III or IV is improving in health or in symptoms enough for it to reach a better NYAH/ISACH class such as class II or even class I, in particular compared to non-treated dogs. The same is valid for a dog being classified according the more recent used lettering system (A, B1, B2, C, & D). Thus, a dog belonging to stage B2 or C is improving in symptoms for it to be graded as stage B1 or B2, respectively, in particular compared to non-treated dogs, or a stage C dog can improve in health to be graded in any of the previous better stages or classes as defined above.

The term “quality of life” as used herein relates also to a tendency of a better quality of life (QoL) when treated with a funny current (I_{f}) inhibitor, in particular cilobradine versus placebo treated dogs. For the purpose of the present invention the dog's quality of life is assessed, in particular by owners, in general as described by Freeman et al. 2005 (Freeman L M et al., Development and evaluation of a questionnaire for assessing health-related quality of life in dogs with cardiac disease, Journal of the American Veterinary Medical Association (2005), Vol: 226, Issue: 11, Pages: 1864-1868. The change, in particular the improvement of the quality of life, is measured with the following grading system: 0 (not at all/none), 1 (very little/very few), 2 (little/few), 3 (moderately/some), 4 (much/many) and 5 (very much/a lot). The assessment further includes an evaluation of a development of and/or changes in breathing difficulties, coughs, wheeze when breathing, tiredness/fatigue/low energy, fainting/collapsing, eating habits, behavior in general, difficulties during recreational pastimes, exercise restriction, the need of sitting/lying down during walks, difficulties in getting comfortable, changing sleeping habits, toilet habits and vomiting.

The dog's quality of life can also be assessed by the owner on a daily base. The change is measured with the following grading system: 1 (better), 2 (equal), 3 (worse). The assessment refers to the overall perception of the owner including all his/her impressions.

Thus, the method for improving the quality of life as mentioned herein in particular relates to an increase in one of the grading systems as described above. As an example the invention relates to improving quality of life in canine patients being grade 3 or 4 to grade 2 or 3 respectively after the administration as described herein, in particular compared to non-treated dog.

Furthermore, the ease of administration also contributes to the quality of life of the animal in need of such treatment as the treatment can only be really successful when it is possible to administer the pharmaceutical composition for the treatment of the herein described heart diseases to the canine, preferably dog. Thus an additional assessment can be made with regard to ease of administration, the majority of the time, whether it was mostly uncomplicated to give, difficult to give or often not possible to give a pharmaceutical composition comprising or consisting of a funny current (I_{f}) inhibitor, in particular cilobradine and other excipients and/or vehicles. In addition the owner is asked if the medication was given in the majority of time with the application syringe directly into the mouth or with a small amount of food.

The term “improving general health condition” as used herein relates to findings during clinical examination, results of the laboratory measurements, assessment of echocardiography, ECG, X-ray parameters, blood pressure measurement, dose of administered medication as well as the dog's behavior. Thus, in order to evaluate the improvement of the general health condition of a canine patient, preferably at least two examinations have to be performed, in particular before and after administration as described herein. As an example the general health condition of a canine patient is improved if the findings during clinical examination, results of the laboratory measurements, assessment of echocardiography, ECG, X-ray parameters, dose of administered medication as well as the dog's behavior show a beneficial effect of the administration as described herein, in particular compared to non-treated dogs.

The term “prolonging life expectancy” is also referred to and encompasses “long-term survival time” or “reducing cardiac mortality or morbidity”, which as used herein relates to the superiority of funny current (I_{f}) inhibitor, in particular cilobradine, to placebo, in particular non-treatment, in dogs suffering from heart diseases as defined above and/or heart failure as defined above, meaning that the dogs have a higher life span following an administration as described herein, in particular on a regular daily basis.

Cardiac mortality is further defined to be an event when a spontaneous death occurs during the study period, where no other clinical cause could be identified despite the knowledge of the underlying cardiac disease. Cardiac morbidity is further defined to be an event when a progression of the cardiac disease occurs which requires rescue therapy (equals Stage D of the lettering system (A, B 1, B2, C, & D). Rescue therapy is given to an already pre-treated dog in the event of cardiac morbidity. Furthermore, cardiac morbidity is defined as progression of the cardiac disease with adjunct worsening of clinical symptoms (e.g. worsening of respiratory signs) beside standard therapy when one of the following criteria below is met: The dog is refractory to standard therapy with e.g. diuretics such as loop diuretics, thiazides, carbonic anhydrase inhibitors, potassium-sparing diuretics, calcium-sparing diuretics or osmotic diuretics, a PDE III inhibitor and possible ACE inhibitor requiring higher diuretic treatment, preferably a diuretic, with dosages above the 6-8 mg/kg once to three times daily to control clinical symptoms (e.g. dyspnoea) of heart failure; the dog requires additional treatment, such as Calcium-channel blockers, β-receptor blocker, diuretics such as a loop diuretic, thiazide diuretic, or potassium-sparing diuretics, or other antiarrhythmic drugs because of worsening of clinical symptoms (e.g. dyspnoea, lethargy) and either worsening of echocardiographic examination parameters (e.g. significantly decreased FS, left atrium size enlargement) compared to baseline assessment or worsening of kidney function or development of clinically significant electrolyte imbalances (i.e. hypokaliaemia, hyponatriaemia); the dog requires repeated thoracocentesis.

“Funny current (I_{f}) inhibitor” or funny current (I_{f}) inhibitors”, also referred to as “I_{f} channel blockers” herein relate to an I_{f} inhibitor including pharmaceutically acceptable salts thereof, which selectively block hyperpolarization-activated cyclic nucleotide-gated channels (HCN) in cardiac conductive tissue, channels responsible for the transmembrane current known as I_{f}. It is through blockade of this current that I_{f} channel blockers are assumed to produce their specific bradycardic effect. HCN channels are widely distributed in the nervous system and in the eye they mediate the current known as Iₕ. The effect of zatebradine and cilobradine on the Iₕ channel has also been investigated (Neuroscience, Vol. 59(2), pp. 363-373, 1994 for zatebradine, and British Journal of Pharmacology, Vol. 125, pp. 741-750, 1998 for cilobradine). The results have suggested that Iₕ can also be blocked by these compounds. Cilobradine in particular reduces the heart rate in the sino-atrial node of the heart by selective blockade of I_{f} channels (“funny channel”) through a direct interaction with predominantly HCN4 channels. This blockage is potent and has a dosage and voltage dependency. Cilobradine is a potent selective bradycardiac agent (SBA), which by means of prolonging the time required for diastolic depolarization, reduces heart rate (HR) without effecting contractility (Braunwald E. Control of myocardial oxygen consumption. Am. J. Cardiol 1971; 27:416-432; Kedem J, Acad B A, Weiss H R. Pacing during reperfusion elevates regional myocardial oxygen consumption. Am. J. Physiol., Heart Circ. Physiol 1990; 259:872-878; Granetzy A, Schwanke U, Gams E, Schipke J D, Effects of a bradycardic agent (DK-AH269) on haemodynamics and oxygen consumption of isolated blood-perfused rabbit hearts. J. Clin. Basic Cardiol 2000; 3:191-196). The negative chronotropic effect of cilobradine leads to a reduction of myocardial oxygen demand, prolongation of the diastolic interval, and increased stroke volume with a subsequent increase of myocardial oxygen supply.

Preferred funny current (I_{f}) inhibitors are cilobradine, anilidine, zatebradine and ivabradine or any of their pharmacologically active salts, preferably cilobradine and zatebradine or any of their pharmacologically active salts, even more preferred is cilobradine or any of its pharmacologically active salts, in particular the hydrochloric acid derivative: cilobradine HCl.

Cilobradine (3-[(N-(2-(3,4-dimethoxy-phenyl)-ethyl)-piperidin-3-yl)-methyl]-(7,8-dimethoxy-1,3,4,5-tetrahydro-2H-3-benzazepin-2-one) and its hydrochloride salt, are disclosed for example in EP-B-0 224 794 and its US counterpart U.S. Pat. No. 5,175,157. As already mentioned cilobradine is also known to have a favorable activity in the treatment and/or prevention of heart failure (see EP-B-1 534 296). Cilobradine, zatebradine and alinidine are also known to have a favorable activity in the treatment and induction of the regression of idiopathic hypertrophic cardiomyopathy (HCM), ischemic cardiomyopathy and valvular hypertrophic heart diseases (see WO 01/78699)

Anilidine [2-(N-allyl-2,6-dichloro-anilino)-2-imidazolidine], is disclosed for example in U.S. Pat. No. 3,708,485, and ivabradine 3-[3-[[[(7S)-3,4-dimethoxybicyclo[4.2.0]octa-1,3,5-trien-7-yl]methyl]methylamino]propyl]-1,3,4,5-tetrahydro-7,8-dimethoxy-2H-3-benzazepin-2-one and its hydrochloride salt, is disclosed for example in EP-B-0 534 859.

Zatebradine [1-(7,8-dimethoxy-1,3,4,5-tetrahydro-2H-3-benzazepin-2-on-3-yl)-3-[N-methyl-N-(2-(3,4-dimethoxy-phenyl)-ethyl)-propane], is disclosed in for example in EP-B-0 065 229 and its US counterpart U.S. Pat. No. 5,516,773. Zatebradine is known to have a favorable activity in the treatment of cardiac insufficiency (see EP-B-0 471 388).

Ivabradine is especially known to have a favorable activity in the treatment of myocardial disorders (from EP-B-0 534 859 or U.S. Pat. No. 5,296,482).

The term “patient” as used herein relates to canine patients, preferably dogs, in particular to a canine suffering from heart disease(s) as defined above. According to another embodiment of the invention the canine patients, preferably dogs, suffer from heart failure due to one or more of the above defined etiologies. According to another aspect of the invention the canine patients, preferably dogs, suffer from arrhythmias as defined above.

Thus, according to the invention the funny current (I_{f}) inhibitor or a pharmaceutically acceptable salt thereof, in particular cilobradine, zatebradine, anilidine or ivabradine, preferably cilobradine or zatebradine, even more preferred cilobradine, is used for treating and/or preventing heart diseases and/or heart failure (HF) in canine patients, preferably dogs. Thus according to the invention the funny current (I_{f}) inhibitors or a pharmaceutically acceptable salt thereof, in particular cilobradine, zatebradine, anilidine or ivabradine, preferably cilobradine or zatebradine, even more preferred cilobradine, is used for treating and/or preventing heart diseases as defined above in canine patients, preferably dogs. According to another embodiment of the invention the funny current (I_{f}) inhibitors or a pharmaceutically acceptable salt thereof, in particular cilobradine, zatebradine, anilidine or ivabradine, preferably cilobradine or zatebradine, even more preferred cilobradine, is used for treating and/or preventing heart failure (HF) due to one or more of the above defined etiologies in canine patients, preferably dogs. According to another aspect of the invention the funny current (I_{f}) inhibitors or a pharmaceutically acceptable salt thereof, in particular cilobradine, zatebradine, anilidine or ivabradine, preferably cilobradine or zatebradine, even more preferred cilobradine, is used for treating and/or preventing arrhythmias as defined above in canine patients, preferably dogs.

According to the invention the canine patient, preferably a dog, is additionally monitored with regard to its overall well-being and mortality. Administration of an I_{f}-channel blocker to a canine patient, in particular to dogs, preferably results in not just the treatment and/or prevention of heart disease(s) as defined above and/or heart failure as defined above but also to an improved quality of life and/or general health condition and/or to a prolonged life expectancy.

Thus, according to a further aspect of the invention it is found by the inventors that the use of a funny current (If) inhibitor or a pharmaceutically acceptable salt thereof, in particular cilobradine, zatebradine, anilidine or ivabradine, preferably cilobradine or zatebradine, even more preferred cilobradine, improves the quality of life of canine patients in particular dogs, preferably suffering from heart diseases as defined above, and/or from heart failure (HF) due to one or more of the above defined etiologies, and/or arrhythmias as defined above.

Furthermore, it is found that a funny current (If) inhibitor or a pharmaceutically acceptable salt thereof, in particular cilobradine, zatebradine, anilidine or ivabradine, preferably cilobradine or zatebradine, even more preferred cilobradine, also improves the general health condition of canine patients, in particular dogs, preferably suffering from heart diseases as defined above, and/or from heart failure (HF) due to one or more of the above defined etiologies, and/or arrhythmias as defined above.

According to a further aspect of the invention the inventors find that the use of a funny current (I_{f}) inhibitor or a pharmaceutically acceptable salt thereof, in particular cilobradine, zatebradine, anilidine or ivabradine, preferably cilobradine or zatebradine, even more preferred cilobradine, prolongs the life expectancy of canine patients, in particular dogs, preferably suffering from heart diseases as defined above, and/or from heart failure (HF) due to one or more of the above defined etiologies, and/or arrhythmias as defined above.

The term “effective amount” as used herein means an amount sufficient to achieve an alleviation of a heart disease as defined above and/or heart failure as defined above in a patient when a funny current (I_{f}) inhibitor is administered at a dosage as described herein. The progress of the therapy can be monitored by standard cardiologic diagnosis, for example, by echocardiography, cardiac catheterization, or cardiac MRI, X-ray, ECG, cardiac biomarkers or cardiac magnetic resonance imaging. The progress of the therapy can also be monitored by clinical symptoms as well as quality of life related parameters as defined above/herein. Furthermore the effective amount also gives the patient, preferably canine patients, even more preferred dogs, an improved quality of life and/or improved general health condition and/or prolonged life expectancy. This progress and improvement of well-being obtained by the therapy can be monitored by the pet owner as well as veterinarian.

### Dosage

The dosage regimen for the compounds of the present invention will, of course, vary depending upon known factors, such as the pharmacodynamic characteristics of the particular agent and its mode and route of administration; the species, age, sex, health, medical condition, and weight of the patient; the nature and extent of the symptoms; the kind of concurrent treatment; the frequency of treatment; the renal and hepatic function of the patient, and the effect desired.

A physician or veterinarian can determine and prescribe the effective amount of the drug required to prevent, counter, or arrest the progress of the disorder. By way of general guidance, the dosage per administration of the active ingredient, preferably of a funny current (I_{f}) inhibitor or a pharmaceutically acceptable salt thereof, in particular cilobradine, zatebradine, anilidine or ivabradine, preferably cilobradine or zatebradine, even more preferred cilobradine, when used for the treatment and/or prevention of diseases as defined above, and/or arrhythmias as defined above, and/or heart failure (HF) due to one or more of the above defined etiologies, will range from 0.001 mg/kg bodyweight to 5.0 mg/kg bodyweight per administration, preferably 0.005 to 4.0 mg/kg bodyweight, 0.01 to 3.5 mg/kg bodyweight, 0.05 to 3.0 mg/kg bodyweight, 0.1 to 2.5 mg/kg bodyweight, preferably 0.1 to 3.0 mg/kg bodyweight, 0.2 to 2.0 mg/kg bodyweight, 0.3 to 1.5 mg/kg bodyweight, 0.3 to 1.0 mg/kg bodyweight or 0.5 to 1.0 mg/kg bodyweight per administration. These dosages should be administered once or twice per day. The treatment is advisable in clinically apparent cases, both in acute as well as in chronic settings. The administration of said dosages preferably also results in one, two or all of the following conditions: an improved quality of life, improved general health condition as well as a prolonged life expectancy.

Thus, according to a further aspect of the present invention, the invention relates to the use of a funny current (I_{f}) inhibitor or a pharmaceutically acceptable salt thereof, in particular cilobradine, zatebradine, anilidine or ivabradine, preferably cilobradine or zatebradine, even more preferred cilobradine, for the preparation of a medicament/pharmaceutical composition for the treatment and/or prevention of a patient, preferably a canine patient, in particular a dog, suffering from heart failure as defined above. According to another aspect, the invention provides the use of a funny current (I_{f}) inhibitor or a pharmaceutically acceptable salt thereof, in particular cilobradine, zatebradine, anilidine or ivabradine, preferably cilobradine or zatebradine, even more preferred cilobradine, for the preparation of a medicament/pharmaceutical composition for the treatment of a patient, preferably a canine patient, in particular a dog, suffering from heart failure due to one or more of the above defined etiologies, and/or arrhythmias as defined above. This use preferably also results in one, two or all of the following conditions: an improved quality of life, improved general health condition as well as a prolonged life expectancy in canine patients, in particular dog, suffering from heart disease as defined above and/or heart failure due to one or more of the above defined etiologies, and/or arrhythmias as defined above.

Preferably the funny current (I_{f}) inhibitor or a pharmaceutically acceptable salt thereof, in particular cilobradine, zatebradine, anilidine or ivabradine, preferably cilobradine or zatebradine, even more preferred cilobradine, is used for treating and/or preventing heart diseases as defined above in a canine patient, in particular a dog, wherein the dosage to be administered is in the range from 0.001 mg/kg bodyweight to 5.0 mg/kg bodyweight per administration, preferably 0.005 to 4.0 mg/kg bodyweight, 0.01 to 3.5 mg/kg bodyweight, 0.05 to 3.0 mg/kg bodyweight, 0.1 to 2.5 mg/kg bodyweight, preferably 0.1 to 3.0 mg/kg bodyweight, 0.2 to 2.0 mg/kg bodyweight, 0.3 to 1.5 mg/kg bodyweight, 0.3 to 1.0 mg/kg bodyweight or 0.5 to 1.0 mg/kg bodyweight per day.

Preferably, the funny current (I_{f}) inhibitor or a pharmaceutically acceptable salt thereof, in particular cilobradine, zatebradine, anilidine or ivabradine, preferably cilobradine or zatebradine, even more preferred cilobradine, is used for treating and/or preventing heart failure due to one or more of the above defined etiologies in a canine patient, wherein the dosage to be administered is in the range from 0.001 mg/kg bodyweight to 5.0 mg/kg bodyweight per administration, preferably 0.005 to 4.0 mg/kg bodyweight, 0.01 to 3.5 mg/kg bodyweight, 0.05 to 3.0 mg/kg bodyweight, 0.1 to 2.5 mg/kg bodyweight, preferably 0.1 to 3.0 mg/kg bodyweight, 0.2 to 2.0 mg/kg bodyweight, 0.3 to 1.5 mg/kg bodyweight, 0.3 to 1.0 mg/kg bodyweight or 0.5 to 1.0 mg/kg bodyweight per administration per day.

According to another aspect, the funny current (I_{f}) inhibitor or a pharmaceutically acceptable salt thereof, in particular cilobradine, zatebradine, anilidine or ivabradine, preferably cilobradine or zatebradine, even more preferred cilobradine, is used for treating and/or preventing arrhythmias as defined above in a canine patient, wherein the dosage to be administered is in the range from 0.001 mg/kg bodyweight to 5.0 mg/kg bodyweight per administration, preferably 0.005 to 4.0 mg/kg bodyweight, 0.01 to 3.5 mg/kg bodyweight, 0.05 to 3.0 mg/kg bodyweight, 0.1 to 2.5 mg/kg bodyweight, preferably 0.1 to 3.0 mg/kg bodyweight, 0.2 to 2.0 mg/kg bodyweight, 0.3 to 1.5 mg/kg bodyweight, 0.3 to 1.0 mg/kg bodyweight or 0.5 to 1.0 mg/kg bodyweight per administration per day.

### Administration

The compounds of this invention can be administered in an oral dosage forms as tablets, capsules (each of which may include sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. According to the invention, funny current (I_{f}) inhibitor or a pharmaceutically acceptable salt thereof, in particular cilobradine, zatebradine, anilidine or ivabradine, preferably cilobradine or zatebradine, even more preferred cilobradine, is to be administered orally or parenterally.

The funny current (I_{f}) inhibitor or a pharmaceutically acceptable salt thereof, in particular cilobradine, zatebradine, anilidine or ivabradine, preferably cilobradine or zatebradine, even more preferred cilobradine, is administered once, twice or three times per day, preferably once or twice, even more preferred once per day.

The funny current (I_{f}) inhibitor or a pharmaceutically acceptable salt thereof, in particular cilobradine, zatebradine, anilidine or ivabradine, preferably cilobradine or zatebradine, even more preferred cilobradine, can be administered alone, but generally will be administered with a pharmaceutical carrier selected on the basis of the chosen route of administration and standard pharmaceutical practice.

### Combined Use

Preferably, the funny current (I_{f}) inhibitor such as cilobradine is administered alone or on top of standard therapy. Preferably, such standard therapy is any diuretic, such as a loop diuretic, thiazide diuretic, or potassium-sparing diuretics. Preferred as diuretic is furosemide. The above mentioned diuretic should be administered in a dose of 1 to 6 mg/kg once to three times daily. It may well be that said diuretic can be completely withdrawn once the patient has been stabilized.

Thus, according to a further aspect the present invention relates to the combined use of a funny current (I_{f}) inhibitor or a pharmaceutically acceptable salt thereof, in particular cilobradine, zatebradine, anilidine or ivabradine, preferably cilobradine or zatebradine, even more preferred cilobradine, with a diuretic such as a loop diuretic, thiazide diuretic, or potassium-sparing diuretics, preferably furosemide, for the treatment of a canine patient, in particular a dog, suffering from heart diseases as defined above and/or heart failure as defined above. Preferably, the funny current (I_{f}) inhibitor or a pharmaceutically acceptable salt thereof, in particular cilobradine, zatebradine, anilidine or ivabradine, preferably cilobradine or zatebradine, even more preferred cilobradine, and a diuretic such as a loop diuretic, or thiazide diuretic, or potassium-sparing diuretics, preferably furosemide, are administered at the dosages described herein.

According to a further aspect the present invention relates to a two phase combination therapy for the treatment of a patient suffering from heart failure comprising in the first phase the administration of a funny current (I_{f}) inhibitor or a pharmaceutically acceptable salt thereof, in particular cilobradine, zatebradine, anilidine or ivabradine, preferably cilobradine or zatebradine, even more preferred cilobradine, in combination with a diuretic such as a loop diuretic, thiazide diuretic, or potassium-sparing diuretics, preferably furosemide, and in the second phase the administration of the funny current (I_{f}) inhibitor or a pharmaceutically acceptable salt thereof, in particular cilobradine, zatebradine, anilidine or ivabradine, preferably cilobradine or zatebradine, even more preferred cilobradine, without using a diuretic.

According to a further aspect the present invention relates to the combined use of a funny current (I_{f}) inhibitor or a pharmaceutically acceptable salt thereof, in particular cilobradine, zatebradine, anilidine or ivabradine, preferably cilobradine or zatebradine, even more preferred cilobradine, with one or two or more pharmaceutically active compounds selected from the group comprising or consisting of calcium channel blocker, β-adrenoreceptor antagonists, positive inotropes, ACE inhibitors, PDE III inhibitors, anti-thrombotic agents and antiarrythmic agents, for the treatment and/or prevention of heart diseases as defined above and/or heart failure as defined above, and/or arrhythmias as defined above in a patient, preferably canine patients, even more preferred dogs. In another aspect, the invention relates to a funny current (I_{f}) inhibitor or a pharmaceutically acceptable salt thereof, in particular cilobradine, zatebradine, anilidine or ivabradine, preferably cilobradine or zatebradine, even more preferred cilobradine, with one or two or more pharmaceutically active compounds selected from the group comprising or consisting of calcium channel blocker, a β-adrenoreceptor antagonists, positive inotropes, ACE inhibitors, PDE III inhibitors, antithrombotic agents and other antiarrythmic agents for the treatment and/or prevention of heart failure due to one or more of the above defined etiologies in a patient, preferably canine patient, even more preferred a dog. The above described combined uses are also useful for one, two or all of the following conditions: improvement of the quality of life, improvement of general health condition as well as a prolongation of life expectancy in canine patients, in particular dogs, suffering from heart disease as defined above and/or heart failure due to one or more of the above defined etiologies, and/or arrhythmias as defined above. The invention relates to the management of heart diseases as defined above in dogs due to the above defined etiologies in conjunction with concomitant therapy, such as for example with diuretics such as a loop diuretic, thiazide diuretic, or potassium-sparing diuretics, preferably furosemide, as needed. Further, the invention relates to the management of heart failure in dogs due to the above defined etiologies in conjunction with concomitant therapy, such as for example with a PDE III inhibitor, or for example a diuretic such as a loop diuretic, thiazide diuretic, or potassium-sparing diuretics, preferably furosemide, as needed.

In a different aspect, one subject of the invention pertains to pharmaceutical compositions comprising cilobradine ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on), cilobradine hydrochloride ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on hydrochloride), zatebradine (1-(7,8-dimethoxy-1,3,4,5-tetrahydro-2H-3-benzazepin-2-on-3-yl)-3-[N-methyl-N-[(2-(3,4-dimethoxyphenyl)ethyl]amino]-propane), or alinidine (2-(N-allyl-2,6-dichloro-anilino)-2-imidazoline) for use in a method for the treatment of a heart disease as defined above, and/or heart failure as defined above and/or arrhythmias as defined above of an animal, preferably a mammal, more preferred a predominantly carnivorous mammal, even more preferred canine, most preferred a dog.

In line with the explanations above, cilobradine or cilobradine hydrochloride are preferred, more preferred is cilobradine hydrochloride. It is particularly preferred to use formulations that are particularly good for administration to dogs for the treatment of heart diseases as defined above, and/or heart failure as defined above, and/or arrhythmias as defined above.

A liquid pharmaceutical composition can have a final concentration of the active ingredient of 0.5 to 20 mg/ml, 0.5 to 10 mg/ml, 0.5 to 5 mg/ml, preferred 0.75 to 4 mg/ml, and more preferred 0.5 to 3 mg/ml. The medical formulation may also be a tablet or granules.

The invention thus further provides the funny current (I_{f}) inhibitor or the pharmaceutically acceptable salt thereof for use in a method as described herein, wherein the funny current (I_{f}) inhibitor is selected from zatebradine (1-(7,8-dimethoxy-1,3,4,5-tetrahydro-2H-3-benzazepin-2-on-3-yl)-3-[N-methyl-N-[(2-(3,4-dimethoxyphenyl)ethyl]amino]-propane), 3-[(N-(2-(3,4-dimethoxy-phenyl)-ethyl)piperidin-3-yl)-methyl]-(7,8-dimethoxy-1,3,4,5-tetrahydro-2H-3-benzazepin-2-on, its enantiomer cilobradine ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on) or alinidine (2-(N-allyl-2,6-dichloro-anilino)-2-imidazoline), mostly preferred cilobradine hydrochloride ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on hydrochloride).

Also, the invention thus provides the funny current (I_{f}) inhibitor or the pharmaceutically acceptable salt thereof for use in a method as described herein, wherein the funny current (I_{f}) inhibitor or the pharmaceutically acceptable salt thereof is contained in a pharmaceutical composition in overall liquid form, comprising or containing the funny current (I_{f}) inhibitor or the pharmaceutically acceptable salt thereof in a final concentration of 0.5 to 20 mg/ml, 0.5 to 10 mg/ml, 0.5 to 5 mg/ml, 0.75 to 4 mg/ml, or 0.5 to 3 mg/ml.

The invention thus also provides the use of a funny current (I_{f}) inhibitor or a pharmaceutically acceptable salt thereof for preparing a medicament, as described herein, wherein the funny current (I_{f}) inhibitor is selected from zatebradine (1-(7,8-dimethoxy-1,3,4,5-tetrahydro-2H-3-benzazepin-2-on-3-yl)-3-[N-methyl-N-[(2-(3,4-dimethoxyphenyl)ethyl]amino]-propane), 3-[(N-(2-(3,4-dimethoxy-phenyl)-ethyl)piperidin-3-yl)-methyl]-(7,8-dimethoxy-1,3,4,5-tetrahydro-2H-3-benzazepin-2-on, its enantiomer cilobradine ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on) or alinidine (2-(N-allyl-2,6-dichloro-anilino)-2-imidazoline), mostly preferred cilobradine hydrochloride ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on hydrochloride).

Also, the invention thus provides the use of a funny current (I_{f}) inhibitor or a pharmaceutically acceptable salt thereof for preparing a medicament, as described herein, wherein the funny current (I_{f}) inhibitor or the pharmaceutically acceptable salt thereof is contained in a pharmaceutical composition in overall liquid form, comprising or containing the funny current (I_{f}) inhibitor or the pharmaceutically acceptable salt thereof in a final concentration of 0.5 to 20 mg/ml, 0.5 to 10 mg/ml, 0.5 to 5 mg/ml, 0.75 to 4 mg/ml, 0.5 to 3 mg/ml or 1 to 2 mg/ml.

The invention thus also provides a method of
treating and/or preventing arrhythmias due to underlying heart diseases such as DCM and MV and/or
treating and/or preventing heart diseases as defined above and/or
treating or preventing heart failure as defined above and/or
improving the quality of life and/or
improving general health condition and/or
prolonging life expectancy
   in a canine patient, preferably a dog, as described herein, wherein the funny current (I_{f}) inhibitor is preferably selected from zatebradine (1-(7,8-dimethoxy-1,3,4,5-tetrahydro-2H-3-benzazepin-2-on-3-yl)-3-[N-methyl-N-[(2-(3,4-dimethoxyphenyl)ethyl]amino]-propane), 3-[(N-(2-(3,4-dimethoxy-phenyl)-ethyl)piperidin-3-yl)-methyl]-(7,8-dimethoxy-1,3,4,5-tetrahydro-2H-3-benzazepin-2-on, its enantiomer cilobradine ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on) or alinidine (2-(N-allyl-2,6-dichloro-anilino)-2-imidazoline), mostly preferred cilobradine hydrochloride ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on hydrochloride).

Also, the invention thus provides a method of
treating and/or preventing arrhythmias due to underlying heart diseases such as DCM and MV and/or
treating and/or preventing heart diseases as defined above and/or
treating or preventing heart failure as defined above and/or
improving the quality of life and/or
improving general health condition and/or
prolonging life expectancy
   in a canine patient, preferably a dog, as described herein, wherein the funny current (I_{f}) inhibitor or the pharmaceutically acceptable salt thereof is contained in a pharmaceutical composition in a tablet, granule or liquid form, comprising or containing the funny current (I_{f}) inhibitor or the pharmaceutically acceptable salt thereof in a final concentration of 0.5 to 20 mg/ml, 0.5 to 10 mg/ml, 0.5 to 5 mg/ml, 0.75 to 4 mg/ml, 0.5 to 3 mg/ml, or 1 to 2 mg/ml.

### BRIEF DESCRIPTION OF THE FIGURES

FIG. 1: This figure shows the positive inotropic effect of cilobradine HCl (0.3 mg/kg bodyweight) administered once daily (light blue) and twice daily (dark blue) in comparison to control (grey).

FIG. 2: This figure shows the positive lusitropic effect of cilobradine HCl (0.3 mg/kg bodyweight) administered once daily (light blue) and twice daily (dark blue) in comparison to control (grey).

### EXAMPLES

The following examples serve to further illustrate the present invention; but the same should not be construed as a limitation of the scope of the invention disclosed herein.

### Example 1

### Formulation of a Compound for the Treatment of a Heart Disease

This example aims at the formulation of compound (+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on hydrochloride in liquid form.

For the production of multi-layered particles for incorporation into a liquid dosage form, a three-step process was applied which process is summarized in table 1.

TABLE 1
Flow chart for the production of multi-layered particles according to
the invention, comprising or containing (+)-3-[(N-(2-(3,4-dimethoxy-
phenyl)ethyl)-piperidin-3-(S)-yl)-methyl]-(7,8-dimethoxy-1,3,4,5-
tetrahydro-2H-3-benzazepin-2-on hydrochloride as active ingredient.

| | starting | | |
|---|---|---|---|
| step | material | Coating | result |
| 1 | inert core | drug layering with pharmaceutically | IR pellets |
| | particles | active ingredient and HPMC/ | |
| | | magnesium stearate | |
| 2 | IR pellets | seal coating with PVP K 30/Talc/ | SC (seal |
| | | colloidal siliciumdioxide | coated) pellets |
| 3 | SC pellets | taste masking coating with EC/ | final multi- |
| | | HPMC/magnesium stearate/ | layered |
| | | colloidal siliciumdioxide | particle |

### Step 1: Drug Layering

Like in example 1 microcrystalline cellulose particles with an average diameter of 100 μm were used as starting material and layered with active ingredient and binder, using water as solvent. The layer material consisted of 66.6% (w/w) of the pharmaceutically active ingredient, 31.7% (w/w) HPMC (PHARMACOAT® 606; see example 1) and 1.7% (w/w) magnesium stearate, dispersed in purified water to yield approx. 19% solids in the spraying liquid.

The resulting composition of the produced IR pellets is given in table 2.

TABLE 2
Composition of IR pellets comprising or containing (+)-3-[(N-
(2-(3,4-dimethoxy-phenyl)ethyl)-piperidin-3-(S)-yl)-methyl]-(7,8-
dimethoxy-1,3,4,5-tetrahydro-2H-3-benzazepin-2-on hydrochloride

| Component | Amount [% (w/w)] |
|---|---|
| inert core particles (CELLETS ® 100) | 80.94 |
| pharmaceutically active ingredient | 12.70 |
| HPMC (PHARMACOAT ® 606) | 6.04 |
| Magnesium stearate | 0.32 |

### Step 2: Seal Coating

The IR pellets produced in step 1 were further processed in the same apparatus by spraying the seal coating onto the IR pellets. The material for the seal coating was composed of PVP K 30 (commercially available by the provider BASF, Ludwigshafen, Germany, under the trade name KOLLIDON® 30/talc, at a weight-percent ratio of 75.4:22.5, dispersed in a 94:6 mixture (m/m) of acetone and ethanol. 0.5% (w/w) of a highly disperse (colloidal) silicium dioxide (AEROSIL® 200, commercially available from Evonik) was added by an additional (intermediate step) to the seal coated material after the application of the PVP K 30/talc mixture and drying of the organic solvent.

The total composition of the SC (seal coated) pellets yielded in this step (cilobradine seal coated pellets) is given in table 3.

TABLE 3
Composition of SC pellets comprising or containing (+)-3-[(N-
(2-(3,4-dimethoxy-phenyl)ethyl)-piperidin-3-(S)-yl)-methyl]-(7,8-
dimethoxy-1,3,4,5-tetrahydro-2H-3-benzazepin-2-on hydrochloride

| Component | Amount [% (w/w)] |
|---|---|
| IR pellets comprising the | 76.54 |
| pharmaceutically active ingredient | |
| PVP K 30 (KOLLIDON ® 30) | 17.67 |
| Talc | 5.29 |
| colloidal silicium dioxide | 0.5 |
| (AEROSIL ® 200) | |

### Step 3: Final Coating (Taste Masking Coating)

The SC pellets produced in step 2 were further processed in the same apparatus by spraying the final coating onto the SC pellets. The material for the final taste and/or odor masking coating was composed of EC/HPMC/magnesium stearate at a weight-percent ratio of 55.2:23.8:19.8 (the ratio of EC/HPMC in the film coating being about 70:30). For this purpose, EC, HPMC and magnesium stearate were dispersed in a 1:1 mixture (v/v) of methanol and dichloromethane, and sprayed onto the SC pellets.

The coating was applied to a thickness of 75% based on the initial amount of SC pellets.

Like in step 2, 0.5% of the colloidal silica of AEROSIL® 200 were added to the final product before sieving.

The chosen EC was ETHOCEL® 45 cps STD Premium, commercially available from Dow Chemical, Schwalbach, Germany. The chosen HPMC for this layer was METHOCEL® ES Premium LV, commercially available from Dow Chemical. AEROSIL® 200 was provided by Evonik.

The overall composition of the final multi-layered particles is given in table 4.

TABLE 4
Composition of final multi-layered particles comprising
or containing (+)-3-[(N-(2-(3,4-dimethoxy-phenyl)ethyl)-
piperidin-3-(S)-yl)-methyl]-(7,8-dimethoxy-1,3,4,5-
tetrahydro-2H-3-benzazepin-2-on hydrochloride

| Component | Amount [% (w/w)] |
|---|---|
| SC pellets comprising the | 56.86 |
| pharmaceutically active ingredient | |
| EC | 23.86 |
| HPMC | 10.25 |
| Magnesium stearate | 8.54 |
| colloidal silicium dioxide | 0.5 |
| (AEROSIL ® 200) | |

Table 5 discloses the detailed overall composition of taste masked multi-layered particles comprising or containing (+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on hydrochloride, as produced according to steps 1-3 of this example, along with the assumed physicochemical function of the respective material.

TABLE 5
Detailed composition of final multi-layered particles
comprising or containing (+)-3-[(N-(2-(3,4-dimethoxy-
phenyl)ethyl)-piperidin-3-(S)-yl)-methyl]-(7,8-dimethoxy-
1,3,4,5-tetrahydro-2H-3-benzazepin-2-on hydrochloride

| | Amount | |
|---|---|---|
| Component | [% (w/w)] | Function |
| inert core particles | 35.23 | Carrier |
| (CELLETS ® 100) | | |
| pharmaceutically active | 5.53 | Drug layer |
| ingredient cilobradine | | |
| (calculated as hydrochloride) | | |
| HPMC | 2.63 | |
| Polyvinylpyrrolidone K 30 | 10.05 | Seal coating |
| Talc | 3.01 | |
| Ethylcellulose | 23.86 | Final coating (taste |
| HPMC | 10.25 | masking) |
| Magnesium stearate | 8.68 | |
| colloidal silicium dioxide | 0.78 | Reduction of electrostatic |
| (AEROSIL ® 200) | | charging in seal coating |
| | | and final coating |

### Dissolution Experiments

The multi-layered particles produced in this example were tested with respect to their dissolution properties in the same way as the particles according to example 1, again at the two different pH values of 6.8 and 1. Measured values are the total percentage of released material after the respective time, normalized to the theoretical drug content.

The result is given in table 6.

TABLE 6
Drug release from multi-layered particles comprising or containing
(+)-3-[(N-(2-(3,4-dimethoxyphenyl)ethyl)-piperidin-3-
(S)-yl)-methyl]-(7,8-dimethoxy-1,3,4,5-tetrahydro-2H-3-benzazepin-
2-on hydrochloride finally coated with EC/HPMC 70:30 at pH
1 and pH 6.8 (mean, n ≧ 3; visualized in FIG. 4)

| Time [min] | % released at pH 1 | % released at pH 6.8 |
|---|---|---|
| 0 | 0.0 | 0.0 |
| 5 | 19.4 | 7.2 |
| 10 | 25.6 | 9.4 |
| 30 | 53.4 | 16.7 |
| 45 | 72.9 | 23.2 |
| 60 | 87.2 | 30.9 |
| 90 | 103.4 | 48.3 |

According to these data, the dissolution from EC/HPMC coated pellets is delayed, therefore providing efficient taste and/or odor masking of the bitter drug (+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on hydrochloride.

Usually, EC/HPMC films show a sustained release behavior that is independent of pH. In this case, however, the product surprisingly showed a slower release at pH 6.8 which is favorable with regards to the invention, i.e. providing efficient taste masking in the oral cavity and a faster release in the acidic stomach. This may be explained by the lipophilicity profile of the active substance cilobradine HCl, which is slightly more lipophilic at neutral pH values. This, together with the coating applied, may have led to a slower release.

### Liquid Pharmaceutical Composition

In order to prepare a liquid pharmaceutical composition, the final multi-layered particles comprising or containing the active ingredient cilobradine prepared in the way explained above were incorporated into an oily liquid. This liquid consisted of a mixture of Medium chain triglycerides (MIGLYOL® 821, bought from Sasol, Hamburg, Germany), a hydrophilic colloidal silicium dioxide (AEROSIL® 200, Evonik), a hydrophobic colloidal silicium dioxide (AEROSIL® R972, Evonik) and meat flavor, at the weight ratios listed in table 7.

TABLE 7
Liquid pharmaceutical composition comprising or containing multi-
layered particles comprising or containing (+)-3-[(N-(2-(3,4-
dimethoxy-phenyl)ethyl)-piperidin-3-(S)-yl)-methyl]-(7,8-dimethoxy-
1,3,4,5-tetrahydro-2H-3-benzazepin-2-on hydrochloride

| | Amount |
|---|---|
| Component | [% (w/w)] |
| medium chain triglycerides | 93.23 |
| (MIGLYOL ® 821) | |
| hydrophilic colloidal silicium dioxide | 4.44 |
| (AEROSIL ® 200) | |
| hydrophobic colloidal silicium dioxide | 1.82 |
| (AEROSIL ® R972) | |
| meat flavor | 0.51 |

The multi-layered particles as produced according to step 3 were suspended in the mentioned liquid composition to an amount of about 3.8% (w/v), resulting in a concentration of 2 mg/ml of the pharmaceutically active ingredient (calculated as hydrochloride).

It has been found that the composition of the oily solvent, especially the mixture of hydrophilic and hydrophobic colloidal silica, ensures an appropriate viscosity behavior of the suspension that remains more or less unchanged over the storage period. During storage, the suspension exhibits a high viscosity, preventing sedimentation of the suspended cilobradine pellets. If shaken, the viscosity of the suspension is transiently lowered so that it can easily be applied via a syringe-like oral dispenser.

### Example 2

### Hemodynamic Effects and Mechanism of Action in Conscious Dogs

Heart rate (HR) and arterial blood pressure (BP) were measured in conscious dogs using telemetry methodology. They received daily oral doses of 0.3 mg/kg over 5 days. HR measured prior drug administration was reduced from 64.0±6.81 beats per minute (bpm) to 50.5±5.54 bpm on day 5. maximal effect on HR were seen between 1.5 and 3.5 hours post-dosing and HR reached values between 45 and 53 bpm. No effect on arterial BP was seen. The study demonstrated that Cilobradine reduces the HR potently in dogs with no direct effect on vascular tone or myocardial contractility.

Cardiovascular and Electrocardiographic Effects of Cilobradine (DKAH 3 CL) in Dogs:

The effect of DKAH 3 CL (0.05 or 0.1 mg/kg, i.v.) on electrocardiogram (ECG) was examined in anesthetized dogs. The dose of 0.05 mg/kg reduced spontaneous HR by 23% whereas 0.1 mg/kg i.v. reduced HR by 42%. Systolic and diastolic pressure was not affected. The effective refractory times of the sinus and atrioventricular (AV) node increased with 0.05 mg/kg by 31% and 30% respectively.

Hyodynamic Action in Conscious, Chronically Instrumented Dogs:

Cilobradine (DKAH 3 CL) was given orally to conscious, chronically instrumented dogs at dose of 2.5 mg/kg. HR dropped by up to 40% while blood pressure was not affected. Left ventricular end-diastolic pressure rose. This effect on preload may be explained by prolongation of the filling period due to prolongation of diastolic duration. Renal blood flow was maintained and coronary blood flow decreased by up to 40% most likely as a consequence of a drop in myocardial oxygen demand.

### Example 3

### A Radiotelemetric Study to Evaluate the Pharmacodynamic Effects of Cilobradine HCl 2 Mg/Ml Oral Suspension after Repeated Once and Twice Daily Oral Administration to Healthy Beagle Dogs

Cilobradine HCl 2 mg/ml oral suspension was given once or twice daily at doses of 0.3 mg/kg bodyweight to conscious, healthy Beagle dogs chronically implanted with a telemetry system. Heart rate, systolic blood pressure, diastolic blood pressure, aortic pressure, left ventricular pressure, electrocardiogram (ECG), and body temperature were recorded. Before each treatment sequence all dogs underwent a 24 hour pre-dosing measurement which served as a baseline (control) measurement. After dosing the dogs were left undisturbed for the duration of the study, which lasted 24 hours, with the exception of the second dosing 10-12 hours after the first treatment.

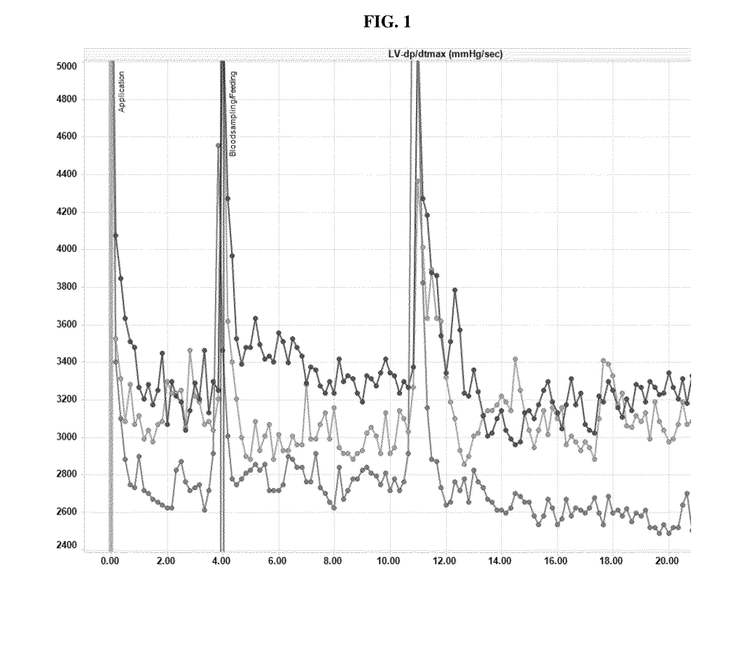

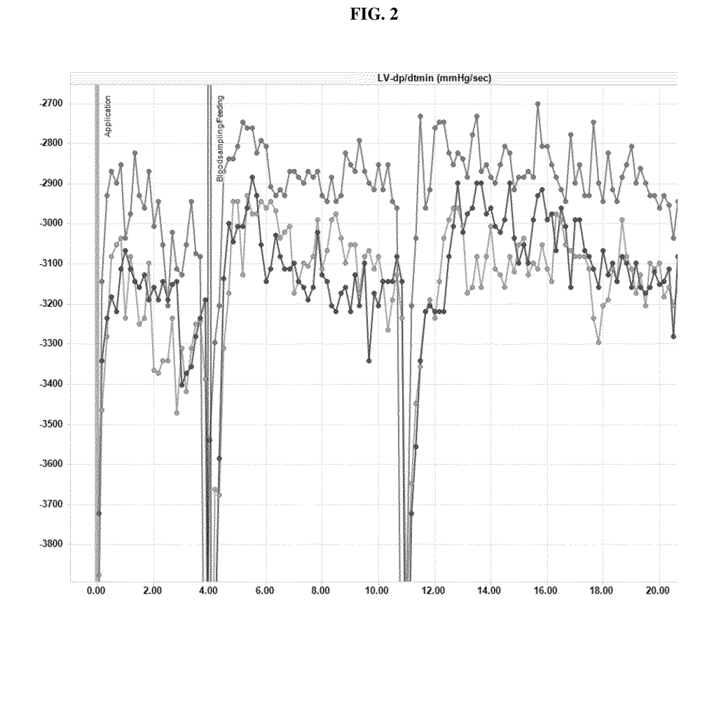

Results from this ongoing study demonstrated that after repeated once and twice daily oral administration of 0.3 mg/kg bodyweight cilobradine HCl to healthy Beagle dogs heart rate decreased by approximately 25% and 30%, respectively. In addition, the maximum of the first derivative of the left ventricular pressure against time (LVdP/dtmax) increased from 2700 mmHg/s to 3000 mmHg/s with 0.3 mg/kg bodyweight cilobradine HCl once daily and to 3550 mmHg/s with 0.3 mg/kg bodyweight cilobradine HCl twice daily, demonstrating a positive inotropic effect of cilobradine HCl (increase of myocardial contractility). Furthermore, the minimum of the first derivative of the left ventricular pressure against time (LVdP/dtmin) was increased from −2700 mmHg/s to −3000 mmHg/s with 0.3 mg/kg bodyweight cilobradine HCl once daily and to −3550 mmHg/s with 0.3 mg/kg bodyweight cilobradine HCl twice daily, reflecting a positive lusitropic effect (increase of myocardial relaxation). In summary, this study revealed that Beagle dogs dosed with 0.3 mg/kg bodyweight cilobradine HCl once or twice daily demonstrated an unexpected combination of a positive inotropic effect (increase of myocardial contractility) (FIG. 1) with a simultaneous positive lusitropic effect (increase of myocardial relaxation) (FIG. 2) despite a decrease in heart rate. This unexpected combined effect is an improved treatment option in dogs with heart diseases since its positive effects on lowering heart rate with simultaneous increase in cardiac contractility and cardiac relaxation leads to an improved overall cardiac function.

1. A funny current (I_{f}) inhibitor or a pharmaceutically acceptable salt thereof for use in a method for treating a canine patient suffering from heart disease.

2. The funny current (I_{f}) inhibitor or a pharmaceutically acceptable salt thereof for use in a method according to claim 1, wherein the canine patient suffers from heart failure.

3. The funny current (I_{f}) inhibitor or a pharmaceutically acceptable salt thereof for use in a method according to claim 1, wherein the canine patient suffers from arrhythmia.

4. The funny current (I_{f}) inhibitor or a pharmaceutically acceptable salt thereof for use in a method according to claim 1, wherein the heart disease is selected from the group consisting of: dilated cardiomyopathy, mitral valve insufficiency, arrhythmia, tachyarrhythmia, arterial arrhythmia, atrioventricular nodal arrhythmia, tachycardia, supra and/or ventricular tachycardia.

5. The funny current (I_{f}) inhibitor or a pharmaceutically acceptable salt thereof for use in a method according to claim 2, wherein the heart failure is due to one or more of the heart diseases selected from the group consisting of: dilated cardiomyopathy, mitral valve insufficiency, arrhythmia, tachyarrhythmia, arterial arrhythmia, atrioventricular nodal arrhythmia, tachycardia, supra and/or ventricular tachycardia.

6. The funny current (I_{f}) inhibitor or a pharmaceutically acceptable salt thereof for use in a method according to claim 3, wherein the arrhythmia is due to one or more heart diseases selected from the group consisting of: dilated cardiomyopathy and/or mitral valve insufficiency.

7. The funny current (I_{f}) inhibitor or a pharmaceutically acceptable salt thereof for use in a method according to claim 1, wherein the method for treating is prevention.

8. The funny current (I_{f}) inhibitor or a pharmaceutically acceptable salt thereof for use in a method according to claim 1, wherein the funny current (I_{f}) inhibitor is selected from the group consisting of cilobradine, zatebradine, ivabradine and anilidine, preferably is cilobradine.

9. The funny current (I_{f}) inhibitor or a pharmaceutically acceptable salt thereof for use in a method according to claim 1, wherein the funny current (I_{f}) inhibitor is to be administered in oral or parenteral form.

10. The funny current (I_{f}) inhibitor or a pharmaceutically acceptable salt thereof for use in a method according to claim 1, wherein the funny current (I_{f}) inhibitor or a pharmaceutically acceptable salt thereof is prepared in a form to be used in a dosage between 0.001 and 5.0 mg active ingredient per kg bodyweight per day.

11. The funny current (I_{f}) inhibitor or a pharmaceutically acceptable salt thereof for use in a method according to claim 1, wherein the funny current (I_{f}) inhibitor or a pharmaceutically acceptable salt thereof is to be administered in a daily dosage between 0.001 and 5 mg/kg bodyweight.

12. The funny current (I_{f}) inhibitor or a pharmaceutically acceptable salt thereof for use in a method according to claim 1, wherein the funny current (I_{f}) inhibitor or a pharmaceutically acceptable salt thereof is to be administered together with diuretics, preferably furosemide, ACE inhibitors, β-blockers, and/or calcium channel blockers.

13. The funny current (I_{f}) inhibitor or a pharmaceutically acceptable salt thereof for use in a method according to claim 1, wherein the canine is a dog.

14. A pharmaceutical composition for use in a method for prolonging life expectancy in a canine patient, wherein such pharmaceutical composition comprises a funny current (I_{f}) inhibitor or a pharmaceutically acceptable salt thereof.

15. The pharmaceutical composition or the pharmaceutically acceptable salt thereof for use in the method according to claim 14, wherein the canine patient is suffering from heart disease.

16. The pharmaceutical composition or the pharmaceutically acceptable salt thereof for use in the method according to claim 14, wherein the canine patient is suffering from heart failure.

17. The pharmaceutical composition or the pharmaceutically acceptable salt thereof for use in the method according to claim 14, wherein the canine patient is suffering from arrhythmia.

18. The pharmaceutical composition or the pharmaceutically acceptable salt thereof for use in the method according to claim 15, wherein the heart disease is selected from the group consisting of: dilated cardiomyopathy, mitral valve insufficiency, arrhythmia, tachyarrhythmia, arterial arrhythmia, atrioventricular nodal arrhythmia, tachycardia, supra and/or ventricular tachycardia.

19. The pharmaceutical composition or the pharmaceutically acceptable salt thereof for use in the method according to claim 16, wherein the heart failure is due to one or more of the heart diseases selected from the group consisting of: dilated cardiomyopathy, mitral valve insufficiency, arrhythmia, tachyarrhythmia, arterial arrhythmia, atrioventricular nodal arrhythmia, tachycardia, supra and/or ventricular tachycardia.

20. The pharmaceutical composition or the pharmaceutically acceptable salt thereof for use in the method according to claim 17, wherein the arrhythmia is due to one or more heart diseases selected from the group consisting of: dilated cardiomyopathy and/or mitral valve insufficiency.

21. The pharmaceutical composition or the pharmaceutically acceptable salt thereof for use in the method according to claim 14, wherein the funny current (I_{f}) inhibitor is selected from the group consisting of cilobradine, zatebradine, ivabradine and anilidine, preferably is cilobradine.

22. The pharmaceutical composition or the pharmaceutically acceptable salt thereof for use in the method according to claim 14, wherein the canine patient is a dog.
